(19) United States
(12) Patent Application Publication
(10) Pub. No.: US 2008/0241202 A1
(43) Pub. Date: Oct. 2, 2008

(54) Multifunctional Cosmetic Composition, Process For Preparing Said Cosmetic Composition and Cosmetic Product

(76) Inventors: Elisangela de Souza Costa, Sao Paulo (BR); Raquel Gomes da Silva, Sao Paulo (BR)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 12/064,550

(22) PCT Filed: Sep. 22, 2006
(86) PCT No.: PCT/BR2006/000193

§ 371 Date: Apr. 3, 2008

(30) Foreign Application Priority Data
Sep. 26, 2005 (BR) PI0503875.8

(51) Int. Cl. A61K 8/06; A61K 8/89

(52) U.S. Cl. 424/401; 424/78.37

(57) ABSTRACT

The present invention relates to a cosmetic composition comprising:—a silicone system composed by at least one light silicone present in an amount ranging from 0.25% to 6.00% by weight, and one heavy silicone present in an amount ranging from 0.25% to 4.00% by weight, and—an emollient system composed by at least one component selected from cupuacu butter, murumuru butter and Shea butter in an amount ranging from 1.0% to 5.0% by weight,—a cosmetically acceptable carrier, all the amounts based on the total weight of the cosmetic composition. This multifunctional cosmetic composition has a rapid absorption, differentiated properties of softness and smoothness, intensive and prolonged moisturizing when applied to the skin and can be applied to the skin before, during and after the bath. Further, the present invention relates to a process for preparing said cosmetic composition and to a cosmetic product comprising said composition.

### FIELD OF THE INVENTION

The present invention relates to a multifunctional cosmetic composition that exhibits rapid absorption, differentiated properties of softness and smoothness, and can be applied to the skin before, during and after the bath and still has an equivalent performance in the cold and in the heat.

Further, the present invention relates to a specific process for preparing said cosmetic composition.

Further, the present invention relates to a cosmetic product that comprises said cosmetic composition.

### DESCRIPTION OF THE PRIOR ART

At present, there is a variety of bath oils available on the market. However, many consumers do not buy this kind of product because they find to be sticky, extremely heavy and to render the skin oily. Further, there are consumers who do not apply moisturizers daily to the skin, because they have not time for this and look for a more practical product, which promotes the sensation of a moisturized and velvety skin during and after the bath.

The applicant will indicate hereinafter some prior-art documents related to the matter of the present invention.

Document U.S. Pat. No. 6,805,871 discloses oil-in-water emulsions comprising emulsifying and film-forming inorganic pigments, which are silicone emulsifiers.

Document U.S. Pat. No. 4,165,385 discloses a water-in-oil emulsion that comprises from 90% to 95% by weight, having 5% of cocoa butter and 5% to 10% of water.

Document U.S. Pat. No. 6,599,513 discloses an emulsion that is bath oil. It comprises vegetable oil and other cosmetically acceptable components.

In this regard, no prior-art document presents teachings relating to the multifunctionality related to the possibility of applying the cosmetic composition at three different moments, having an equivalent performance, coupled to the formation of a film and a sensorial improvement.

### SUMMARY OF THE INVENTION

The present invention has the objective of providing a multifunctional cosmetic composition, being particularly an oil-in-water emulsion that comprises:

a silicone system composed of at least one light silicone present in an amount ranging from 0.25% to 6.00% by weight, and one heavy silicone present in an amount ranging from 0.25% to 4.00% by weight, and

an emollient system composed by at least one component selected from cupuaçu butter, murumuru butter and shea butter in an amount ranging from 1.0% to 5.0% by weight and

a cosmetically acceptable carrier, all the amounts being based on the total weight of the cosmetic composition.

The present invention further has the objective of providing a process of preparing the cosmetic composition in question, which comprises the following steps:

### Preparation of Phase A

adding to a main recipient a cosmetically acceptable carrier;

### Preparation of Phase D

initiating the heating until a temperature ranging from 75° C. to 80° C. is reached;

### Preparation of Phase E

with the temperature between 75° C. and 80° C., solubilizing at least one of the butters. Keeping this temperature, under a high stirring ranging from 30 rpm to 3000 rpm until total homogenization is achieved;

### Preparation of Phase F

initiating the cooling until a temperature between 55° C. and 60° C. is reached and adding at least one light silicone and one heavy silicone.

The present invention further has the objective of providing a cosmetic composition obtainable by the process described above.

### DETAILED DESCRIPTION OF THE INVENTION

The cosmetic composition of the present invention is multifunctional and exhibits rapid absorption, differentiated properties of softness and smoothness and can be applied to the skin before, during and after the bath and still has an equivalent performance in the cold and in the heat.

The cosmetic composition of the present invention comprises associating silicone agents and butters to obtain a variety of formulations with various functionalities for skin care and protection. These properties are still potentiated if the cosmetic composition of the present invention is prepared in accordance with the process described hereinafter.

It has been found that each of the components of this base, either alone or in combination, has a quite advantageous purpose for skin care, namely:

the present composition comprises a high degree of silicones, without, however, causing an oily aspect when applied to the skin. This property is due to the balanced amounts of light and heavy silicones added to the composition of the present invention;

further, the present composition comprises butters actuating as emollients. Thus, the moisturizing provided on the skin is intense and prolonged;

this combination provides high viscosity and a bright appearance;

the combination of the silicone and emollient system comprised in the cosmetic composition of the present invention provides optimization of the properties of softness, smoothness, spreadability and absorption, besides resulting in various possibilities of application of this product to the skin. Thus, the cosmetic composition in question provides softness, comfort and well-being during and after application;

the association of the light and heavy silicones with at least one butter in the amounts established confers to the cosmetic composition of the present invention its multifunctionality, since:

during the pre-bath massage it has excellent spreadability;

when applied during the bath it provides the feeling of skin treatment, film formation and moisturizing; and

when applied as a moisturizer to dried areas or dry skins it provides moisturizing;

the associated silicones promote ease of absorption of the emulsion in the skin and, at the same time, good spreadability, without promoting an oily sensorial;

the butters promote high consistency and heavy oily sensorial, which after rinsing leaves the film formation and moisturizing potential, besides being emollients capable of replacing the natural oil of the skin;

the associated silicones and butters exhibit a synergistic effect that confers to the product the multifunctionality, since it promotes a pleasant feeling in all the application modes.

Tests were made without the butters and without the silicones in the beginning of the study, but it was observed a huge sensorial difference, since either too oily products (with butter alone) or too light products (with silicones alone) were obtained.

The main examples of products that can be prepared by starting from the cosmetic composition of the present invention, which is an oil-in-water type emulsion for daily or seasonal use, are:
Bath oils;
Bath creams;
Body moisturizers
Daily-use face moisturizers;
Night-use face moisturizers;
Body or face cosmetic preparations for children;
Face cosmetic preparations for after-shave use;
Localized-action cosmetic preparations, as for example, for treating
spots, dark circles or puffiness under eyes;
Body and face sunscreens;
Cosmetic preparations for massage;
Cosmetic preparations for treating striae in the skin and cellulite.

The cosmetic composition of the present invention has a variety of desired advantages and characteristics in a cosmetic product for the skin, some of which are listed below:

1. significant improvement of the sensorial aspects;
a. oily and/or sticky texture replaced by a silky, soft, smooth and velvety aspect during and after application of the preparation;
b. difficult spreadability and slow absorption give rise to easy application and spread, high spreadability and absorption in ideal time;
c. unpleasant and uncomfortable sensation at the moment of and after application of the preparation is replaced by a feeling of well-being and pleasure;

2. an intense and long-lasting moisturizing effect from the moment of application until after application to the skin;

3. the cosmetic composition of the present invention does not have negative effects present in compositions having high concentrations of oils, silicones and emollients on the spreadability and stickiness characteristic of the composition;

4. in addition, since the cosmetic composition of the present invention comprises silicones and emollients in specific amounts, it exhibits:

high stability;

promotion of long-lasting moisturizing properties;

pleasant sensation upon application.

5. The present invention is multifunctional, that is to say, it can be applied to the skin before, during and after the bath, promoting moisturizing during the same period of time;

6. the process of preparing the present invention presents solutions of the deficiencies observed today in the preparation of the cosmetic compositions that confer a high level of moisturizing available on the market, namely:

the water phase and oil phase prepared do not form particulate substances (solids in the form of crystal) or waxy mass, and do not promote polymerization reactions;

optimized process, without the need for parallel phases in other equipments;

keeps the emollient power inherent in the emollients chosen and present in the composition;

7. the present invention can be used both in the cold and in the heat, having an equivalent performance;

8. the present invention presents the formation of a film when applied to the skin, even after rinsing, resulting in a prolonged moisturizing;

9. this cosmetic composition is indicated for personal care and “spa moments”;

10. it presents a balanced combination of components that enable high stability;

11. it is an excellent substitute for bath oils and moisturizers, since it provides the same benefits in a single product;

12. it provides a light refreshing effect when applied to the skin;

13. it has essential oil (present in the fragrance) that pleases the senses, promoting a unique moment of relaxation and spa;

14. it promotes softness and moisturizing to the skin, with excellent spreadability and ease of application;

15. as to the moisturizing properties, one can state that the structures observed in the liquid crystals are capable of retaining water in a more effective manner, minimizing its evaporation and promoting a substantial and long-lasting effect.

### Cosmetic Composition of the Present Invention

As already said before, the cosmetic composition of the present invention comprises:

a silicone system composed by at least one light silicone in an amount ranging from 0.25% to 6.0% by weight and one heavy silicone present in an amount ranging from 0.25% to 4.0% by weight, and

an emollient system composed by at least two components selected from cupuaçu butter, murumuru butter and shea butter in an amount ranging from 1.0% to 5.0% by weight,

all the amounts are based on the total weight of the cosmetic composition.

Preferably, this cosmetic composition is an oil-in-water emulsion, being indicated for daily sue.

The emulsion is preferably from 39 to 45% of oil phase and from 55 to 61% of water phase.

In the table below the preferably used proportions of the components present in the cosmetic composition of the present invention are designated. The combination of these components, in amounts corresponding to the proportions defined in the table given below, provides a significant improvement with respect to the compositions known at present. These improvements are perceived with regard to the sensorial aspects (softness, smoothness, prolonged moisturizing, ease of sliding) at the moment of application, formation of a film over the skin and also with regard to aspects related to the moments when this composition can be applied to the skin, being characterized as multifunctional.

| | Preferred proportions (parts by |
|---|---|
| Category | weight) |
| Silicone system | 4 to 10 |
| Emollient system | 5 to 12 |
| Emulsifying system | 3 to 5 |
| Wetting system | 5 to 10 |

The cosmetic composition of the present invention comprises components that are kinetically stable for a long period of time. Further, the addition of the preferred emulsifiers, polymers and stabilizers, cited hereinafter, prolong the stability time of the finished product (which is also dependent upon the preparation process described hereinafter). Further, the cosmetic composition of this invention may receive a high concentration of dyes, which is generally difficult to stabilize. However, in this invention the color remains stable (studies indicate a 2-year validity of the finished product).

### Silicone System

Silicones exhibit emollient and skin conditioning properties.

The silicone system is constituted by at least one light silicone and one heavy silicone. The amount of this system in the composition ranges preferably from 4.0% to 10.0% by weight, based on the total weight of the cosmetic composition of the present invention. This system may further receive optional silicone agents that will be defined hereinafter.

By preference, the silicone system is constituted by cyclomethicone, the silicone being light, and a mixture of cyclomethicone and dimethiconol, the silicone being heavy.

These are regarded as preferred, because they provide, in addition to the characteristics already described, a velvety look to the skin. Cyclomethicone exhibits, in the present composition, a highly dry touch, potentiating the rapidity of absorption of the composition in the skin and not conferring stickiness to the product. On the other hand, the mixture of cyclomethicone and dimethiconol, the latter being an emollient silicone of a less dry touch, thus being oilier, provides a long-lasting emollient effect and still a marked sensorial to the skin.

Alternatively, other light and heavy silicones may be used, as long as they comprise chains equivalent to those cited above. Some examples of silicone that may be added to the cosmetic compositions of the present invention are volatile and non-volatile silicone oils such as, for example, alkyldimethicones, dimethicone copolyols, phenyl trimethicones, caprylyl trimethicones, aminofunctional silicones, phenyl modified silicones, phenyl trimethicones, alkyl modified silicones; polydimethyl silicone oil and combinations thereof or silicone elastomers like cyclomethicone and dimethicone crospolymer, vinyl dimethicone and dimethicone crospolymer, dimethicone and dimethicone crospolymer and cyclopentasyloxane and dimethicone crospolymer.

Preferably, one may add to the present composition dimethicones, phenyl trimethicones, volatile silicones, among others.

### Emollient System

The function of the emollients in cosmetic compositions is to add or replace natural oils on the skin, for the purpose of maintaining the integrity of the hydrolipidic mantle of the skin.

Preferably, the emollient system is constituted by at least one butter selected from cupuaçu butter, murumuru butter and shea butter in an amount ranging from 1.0% to 5.0% by weight, based on the total weight of the cosmetic composition. More preferably, one adds cupuaçu butter and murumuru butter in the amounts cited above, which are highly oily, has low spreadability, which causes the association thereof with other components to render the cosmetic composition a product easy to apply to the skin, keeping it moisturized through the formation of a film and creating three moments of application of the composition to the skin.

In addition, the butters aid in conferring consistency to the product, provide brightness and aid in moisturizing the skin, especially when associated with some moisturizing agent that may be present in the formulation.

Optionally, other emollients may be added to the present composition. Some compounds that may be used as emollients are natural oils, esters, ethers and combinations thereof.

From emollients with low spreadability to emollients with high spreadability may be added, preferably non-comedogenic ones, in order not to cause irritation to the skin.

As high-spreadability emollients, the preferred ones are dicaprylic ether, decyl oleate and cetyl lactate, since they exhibit pleasing sensorials and help to facilitate the sliding when the product is applied to the skin, and still act on the rapidity of absorption of the composition in the skin.

Other emollients that may be added to the composition of the present invention are: conventional lipids as for instance, oils, waxes and other water-insoluble components and polar lipids that are those modified so as to increase their solubility in water by esterification of a lipid to a hydrophilic unit as, for example, hydroxyl groups, carbonyl groups, among others. Some compounds that may be used as emollients are natural oils derived from plants, esters, polyunsaturated fatty acids, lanolin and derivatives thereof. Some natural oils that may be used are derived from damson oil, sesame, soy, groundnut, coco-nut, olive, cocoa-butter, almond, avocado, carnauba, cotton seed, rice bran, peach seed, mango seed, jojoba, macadamia, coffee, grape seed, pumpkin seed, among others and mixtures thereof.

Some ethers and esters may also be used in the function of emollients, such as for example, C₈-C₃₀ alkyl esters of C₈-C₃₀ carboxylic acids, C₁-C₆ diol monoesters and C₈-C₃₀ carboxylic acid diesters, C₁₀-C₂₀ alcohol sucrose monoesters and combinations thereof. Examples of these compounds are dicaprylic ether, isopropyl palmitate, dicaprylyl carbonate, C₁₂₋₁₅ alkyl benzoate, isopropyl isononate, sucrose palmitate, sucrose oleate, isostearyl lactate, glyceryl behenate, triglycerol-4 isostearate, lauryl pyrrolidone carboxylic acid, pantenyl triacetate and combinations thereof.

One may still use other fatty alcohols, mono-, di- or triglycerids ethers that have a lipophilic nature such as dicaprylylic ether, besides synthetic and natural hydrocarbons, organic carbonates like dicaprylyl carbonate and mixture thereof.

In addition, one may use various natural compounds as emollients, as for example, microcrystalline wax, carnauba wax, beeswax, ozokerite wax, among others.

In the preferred embodiments of the present invention, one uses optional emollients, when present, a combination of dicaprylic ether, decyl oleate and cetyl lactate in an amount ranging preferably from 5.0% to 12.0% by weight, based on the total weight of the cosmetic composition of the present invention.

In addition to the components already cited, the cosmetic composition of the present invention may further comprise agents that have specific functions required for each composition necessary for each situation, such as chelating agents, thickening agents, pH-adjusting agents, preservative agents, moistening agents, conditioning agents, other emollients, filmogenic agents, oiliness adsorbing agents, actives, among others. This composition may be called a cosmetic base, since it may receive various optional components in its constitution.

Hereinafter it is listed the optional agents that may be added to the systems comprised in the cosmetic composition of the present invention:

### Refreshing Agent:

One may add to the present composition, as a refreshing agent, mentol in an amount ranging from 0.10% by weight, based on the total weight of the composition, to provide slight refreshment to the skin, since when in contact with water it provides a slight refreshing effect, being highly indicated for use in the summer.

This component provides, when present in the composition of the present invention, a difference in relaxation and spa treatment.

### Emulsifying Agent

As optional emulsifying agents, one may use anionic emulsifiers, non-ionic emulsifiers and polymeric emulsifiers.

Some examples of emulsifying agents may be added: components of various categories of substances such as anionic emulsifiers, cationic emulsifiers, preferably non-ionic ones, such as propoxy and/or ethoxylated fatty alcohols, sorbitan esters, methyl glucose, propylglyceryl glucose, fatty acids and glycols, fatty acids and sucrose, fatty acids and pentaerythritol, either ethoxylated and/or non-ethoxylated, copolymers of ethylene oxide and propylene oxide, alkyl glycosides and polyglicosids, ethoxylated and/or non-ethoxylated animal and vegetable sterols, preferably ethoxylated stearylic alcohols and ethoxylated esters.

In the preferred embodiments, it is added to the present composition an emulsifying system constituted by at least estearet-2, estearet-21 and glyceryl stearate. The amount of this system in the composition ranges preferably from 3.0% to 5.0% by weight, based on the total weight of the cosmetic composition of the present invention.

The combination of the emulsifiers estearet-2 and estearet-21 forms liquid crystals that constitute an intermediate state between the solid and liquid states, called mesophase or mesomorphic state. The formation of these structures varies in function of the concentration of the emulsifiers used, as well as of the temperature and the velocity of the cooling/heating employed in the process. The network of liquid crystals formed provides retention of water close to the skin, resulting in a longer-lasting and prolonged moisturizing.

### Moistening Agent

The function of the moistening agent in the cosmetic composition of the present invention is to provide water retention in the user's skin, that is to say, to provide water to the skin and also prevent loss of water from the skin. The moistening agent still aid in enhancing the efficacy of the emollient reduces scaling of the skin and improves the sensorial of the skin.

Some examples of moistening agents that may be added to the cosmetic compositions of the present invention are: alkylene polyols and derivatives thereof, glycerol, ethoxylated glycerol, propoxylated glycerol, sorbitol, hydroxypropyl sorbitol, among others, C₃-C₆ diols and triols, Aloe Vera extract, butyleneglycol, sugars and starches and derivatives thereof, as for example, alkoxylated glycose, hyaluronic acid, glycolic acid, lactic acid, glycolic acid and salicylic acid, panteol, urea, ethoxylated nonyl phenol, natural oils such as pine oil, oils and waxes and mixtures thereof.

In the preferred embodiments, it is added, as a moistening agent, vegetable bi-distilled glycerin in an amount ranging preferably from 5.0% to 10.0% by weight, based on the total weight of the cosmetic composition of the present invention.

### Carrier

Water is the base of several possibilities of cosmetic compositions acting as a carrier for the other components. The compositions of the present invention comprise preferably demineralized or distilled water in a suitable (q.s.p.) percentage for reaching 100% of the formula, based on the total weight of the present composition. Naturally, other cosmetically acceptable carriers may be used in the present invention.

### Other Optional Components

In order to confer to the cosmetic composition of the present invention some desirable characteristic that has not yet been achieved with components already cited, it may be added optional components that are compatible with its properties. Some of these components that may be added to the composition are:

chelating agents like disodium EDTA;

suspending thickening agent like Carbomero-980;

antioxidant agent like BHT;

neutralizing agent like sodium hydroxide;

polymeric consistency agent like polyacrylamide and C₁₃-₁₄ isoparaffin;

bacteriostatics, bactericides or antimicrobials;

dyes;

plant extracts: chamomile, rosemary, thyme, calendula, carrot extract, common juniper extract, gentian extract, cucumber extract;

preserving agent like phenoxyethanol;

fragrance; and

other cosmetically acceptable components that are compatible with the base composition.

### Process for Preparing the Cosmetic Composition of the Present Invention:

The process for preparing the cosmetic composition of the present invention is described hereinafter. This emulsification process (which uses a temperature ranging from 70° C. to 809° C.) is applicable in the present invention because it ensures the achievement of final compositions that exhibit special physicochemical properties, as for example, optimum physicochemical stability.

Knowing that the obtainment of the multifunctional composition described herein may vary depending on the type and on the concentration of the components used, as well as on the temperature employed in the process (intensity and heating/cooling time), it is important to point out that all the steps described hereinafter should be carried out in order for guaranteeing the desired technical effects.

The steps of the process for preparing the cosmetic composition of the present invention will be presented later. Some parameters used in carrying out said steps are pointed out below:

the stirring of the cosmetic composition in all the steps should be constant;

in this way, in all the steps there should be a stirring mean, that is to say, some kind of mixer is always used;

this mixer may be: a stirrer, an anchor, a scraper, naval stirrer, a rotor/stator homogenizer, a turbine, a combination of these or still any other mean that is capable of keeping the cosmetic composition in question under agitation. Preferably it is used the combination of a rotor/stator-type homogenizer, a stirrer and a scraper; and

eventually it may be used a vacuum system.

The present invention has, as its main characteristic, taking care during the preparation process, which comprises the following steps:

### Preparation of Phase A

adding to a main recipient a cosmetically acceptable carrier;

### Preparation of Phase D

initiating the heating until a temperature raging from 75° C. to 80° C. is reached;

### Preparation of Phase E

with the temperature between 75° C. and 80° C., solubilizing at least one of the butters from cupuaçu butter, murumuru butter and shea butter. Keeping this temperature, under a high stirring ranging from 30 rpm to 3000 rpm until total homogenization is achieved;

### Preparation of Phase F

initiating the cooling until a temperature between 55° C. and 60° C. is reached and adding at least one light silicone and one heavy silicone.

Optionally, one may prepare other phases:

### Preparation of Phase B:

adding a suspending thickening agent at a temperature of about 25° C., mixing the composition at a frequency ranging from 2500 to 3500 rpm until total solubilization of the polymer is achieved;

### Preparation of Phase C:

at a temperature of 25° C., adding a moistening agent;

### Preparation of Phage G:

in a separate recipient, solubilizing a neutralizing agent in the carrier, and pouring onto the main recipient, thus neutralizing the emulsion, at a temperature between 50° C. and 55° C., reaching total homogenization and neutralization;

### Preparation of Phase H:

upon reaching the temperature of 45° C., adding the polymeric consistency agent to form a cream gel;

### Preparation of Phase I:

upon reaching a temperature between 30° C. and 35° C., adding at least one preserving agent, a fragrance and at least one dye. Keeping stirring until the temperature of 25° C. is reached.

An example of a process of preparing the cosmetic composition of the present invention is described hereinafter:

### Preparation of Phase A:

adding to a reactor a cosmetically acceptable carrier and a chelating agent, stirring with a naval propeller, a scraper and homogenizer until complete homogenization is achieved;

### Preparation of Phase B:

adding a suspending thickening agent at a temperature of about 25° C., mixing the composition at a frequency ranging from 2500 to 3500 rpm until total solubilization of the polymer is achieved;

### Preparation of Phase C

at a temperature of 25° C., adding a moistening agent;

### Preparation of Phase D

initiating the heating until a temperature ranging from 75° to 80° C. is achieved, at a stirring frequency of about 2000 rpm;

### Preparation of Phase E

with the temperature between 75° C. and 80° C., solubilizing emulsifying agents, emollient agents and an antioxidant agent. Keep this temperature, under high stirring (naval:—3 rpm, scraper—30 rpm and homogenizer—3000 rpm) for about 20 minutes or until fusion of the solids.

### Preparation of Phase F

initiating the cooling until a temperature between 55° C. and 60° C. is reached and adding silicone systems.

### Preparation of Phase G

in a separate recipient, solubilizing a neutralizing agent in the carrier, and pour onto the main recipient, thus neutralizing the emulsion, at a temperature between 50° C. and 55° C., achieving total homogenization and neutralization;

### Preparation of Phase H

when the temperature of 45° C. is reached, adding a polymeric consistency agent to form a cream gel;

### Preparation of Phase I

when a temperature between 30° C. and 35° C. is reached, adding at least one preservative agent, a fragrance and at least one dye. Keeping the stirring until the temperature of 25° C. is reached.

### Example of the Cosmetic Composition of the Present Invention

The example below is a preferred embodiment of the cosmetic composition of the present invention and should not be interpreted as limiting it. Therefore, many other variations of a composition may be carried out within the protection scope delimited by the claims.

This composition has been carried out by using the process described before. In this regard, one arranges the phases in which each of the components was added to the composition.

Phase A: it was added to the reactor demineralized water and di-sodium EDTA, stirred with a navel propeller, scraper and homogenizer until total solubilization.

Phase B: it was added Carbômero 980, at a temperature of about 25° C., mixed at a frequency ranging from 2500 to 3500 rpm for a period of 10 minutes;

Phase C: still at the temperature of 25° C., it was added vegetable bi-distilled glycerin, the stirring was maintained.

Phase D: the heating of the system was initiated at a stirring frequency of about 2000 rpm, until the temperature of 75-80° C. was reached.

Phase E: with a temperature between 75° C. and 80° C., estearet-2, estearet-21, glyceryl stearate, decyl oleate, dicaprylic ether, cetyl lactate, cupuaçu butter, murumuru butter and BHT were solubilized. The temperature was maintained and the stirring was raised (naval—30 rpm, scraper—30 rpm and homogenizer—3000 rpm) for about 20 minutes or until total fusion of the solids.

Phase F: the cooling was initiated, the specified stirrings were maintained, since the faster the stirring frequency the greater the brightness of the emulsion. When the temperature reached a value between 55° C. and 60° C., cyclomethicone, cyclomethicone and dimethiconol were added and stirred.

Phase G: in a separated recipient, sodium hydroxide was solubilized in water and poured onto the main recipient, thus neutralizing the emulsion, at a temperature between 50° C. and 55° C., under constant stirring, in order for total homogenization and neutralization to take place.

Phase H: with a temperature of 45° C., a polymeric consistency agent was added and maintained the stirring.

Phase I: with a temperature between 30° C. and 35° C., phenoxyethanol, iodopropinyl Butylcarbamate, mentol, the fragrancy and the dyes were added. The stirring was maintained until the temperature of 25° C. was reached.

### EXAMPLE

### Bath Mask

| Phase | Component | Mass amount (%) |
|---|---|---|
| A | Demineralized water | 47.10 |
| A | di-sodium EDTA | 0.10 |
| I | Mentol | 0.10 |
| I | Fragrance | 2.00 |
| I | Dyeing solutions | 10.02 |
| B | Carbomero 980 | 0.05 |
| C | Vegetable bi-distilled | 5.00 |
| | glycerin | |
| E | Cetyl alcohol | 5.50 |
| E | Decyl oleate | 2.00 |
| E | Dicaprylic ether | 2.50 |
| E | Glyceryl stearate | 2.50 |
| E | Stearet-21 | 1.00 |
| E | Cupuacu butter | 2.00 |
| E | BHT | 0.05 |
| E | Murumuru butter | 2.00 |
| E | Stearet-2 | 3.00 |
| E | Cetyl lactate | 2.00 |
| F | Cyclomethicone | 4.00 |
| F | Cyclomethicone and | 2.00 |
| | dimethiconol | |
| G | Demineralized water | 5.27 |
| G | Sodium hydroxide | 0.01 |
| H | Polyacrylamide and | 0.70 |
| | C₁₃-₁₄ isoparaffin and | |
| | lauret-7 | |
| I | Iodopropinyl | 0.20 |
| | butylcarbamate | |
| I | Phenoxyethanol | 0.90 |

### Tests of the Cosmetic Composition of the Present Invention

The composition used in the tests described hereinafter is the one defined in the Example described above (bath mask).

### Test 01—Evaluation of Film Formation

This test has the objective of evaluating the efficacy of the cosmetic composition of the present invention applied to the skin, for the attribute of formation of a film by means of the objective technique (optical microscopy). The reflected-light optical microscopy is capable of measuring surface macroalterations.

It were prepared 9 standardized substrates that mimetize some properties of the human skin. The whole experiment was carried out in an air-conditioned environment.

The samples were previously moisturized, receiving massage for 30 seconds, followed by rinsing for 30 seconds, drying in a stove at 35° C. for 1 hour, whereby the material to be evaluated was obtained (3 different regions being selected for this purpose).

It was verified that this cosmetic composition, when applied onto the substrate, has a single phase.

The basal image of the samples was divided into two phases: high relief and low relief. The filmogenic agents stand out for the low-relief regions, seeking adsorption routes, forming new dark phases (high relief). These coverings, when summed give rise to the formation of a superficial, non-uniform and discontinuous microfilm, indicating their covering power.

### Test 02—Evaluation of Moisturizing

The objective of this study was to evaluate the surface moisturizing layer of the skin after application of the composition according to the invention onto the forearm region of 20 volunteers. The skin moisturizing measurement was made by the Corneometry technique.

It were selected 20 women having dry skin, the volunteers having been instructed to discontinue the use of any cosmetic applied to the forearms 48 hours before the beginning of the test. In each volunteer it were demarcated three sites of 2.5×4.0 cm, called s1, s2 and s3, and in the demarcated sites it were applied at random 20 μL of the composition of the invention or of a Standard Product with known moisturizing data, one of the sites being used as control (without application of any products). The data of code correlation of volunteer/product used are given in the table below

| Volunteer Code | S1 | S2 | S3 |
|---|---|---|---|
| 01 | C | A | |
| 02 | C | A | P |
| 03 | A | C | |
| 04 | C | A | |
| 05 | A | C | |
| 06 | C | A | |
| 07 | A | C | |
| 08 | C | A | P |
| 09 | A | C | |
| 10 | C | A | |
| 11 | A | C | |
| 12 | A | C | |
| 13 | A | C | |
| 14 | C | A | |
| 15 | A | C | |
| 16 | C | A | |
| 17 | A | C | |
| 18 | C | A | |
| 19 | A | C | |
| 20 | C | A | |

Wherein:
A = Composition according to the invention
P = Standard Product
C = Control (site without application of product)

The volunteers remained in an air-conditioned room (22±2° C. and 50±5% UR) throughout the test. It were made basal measurements, after 2 h, 5 h and 8 h from application of the products, according to the tables below.

Basal measurements

| 01 | | | 02 | | | 03 | | | 04 | | | 05 | | |
|---|---|---|---|---|---|---|---|---|---|---|---|---|---|---|
| S1 | S2 | S3 | S1 | S2 | S3 | S1 | S2 | S3 | S1 | S2 | S3 | S1 | S2 | S3 |
| 32.9 | 29.6 | | 41.3 | 40.3 | | 39.4 | 34.5 | | 34.6 | 30.0 | | 31.5 | 27.2 | |
| 33.6 | 28.9 | | 41.6 | 42.5 | | 35.2 | 35.0 | | 35.6 | 36.5 | | 31.1 | 28.2 | |
| 36.6 | 32.4 | | 40.7 | 43.0 | | 36.3 | 36.7 | | 32.0 | 34.8 | | 27.6 | 29.6 | |
| 35.3 | 35.0 | | 44.2 | 42.7 | | 34.4 | 35.0 | | 36.2 | 34.0 | | 27.9 | 28.8 | |
| 33.8 | 27.0 | | 42.5 | 44.5 | | 36.2 | 33.8 | | 31.7 | 31.2 | | 29.5 | 29.3 | |
| | 28.2 | | | | | | | | 32.7 | 32.3 | | | | |
| | 32.5 | | | | | | | | | 33.3 | | | | |
| | 32.2 | | | | | | | | | 37.5 | | | | |
| | 30.2 | | | | | | | | | 30.8 | | | | |
| | 31.0 | | | | | | | | | 32.5 | | | | |
| 06 | | | 07 | | | 08 | | | 09 | | | 10 | | |
| S1 | S2 | S3 | S1 | S2 | S3 | S1 | S2 | S3 | S1 | S2 | S3 | S1 | S2 | S3 |
| 32.0 | 28.6 | | 29.3 | 26.4 | | 40.1 | 36.0 | 38.8 | 37.3 | 34.9 | | 36.2 | 36.2 | |
| 30.9 | 28.0 | | 32.3 | 25.3 | | 39.9 | 38.0 | 34.4 | 38.0 | 32.0 | | 41.7 | 36.3 | |
| 27.9 | 29.3 | | 33 | 23.6 | | 36.9 | 35.3 | 38.3 | 35.7 | 36.7 | | 35.4 | 37.1 | |
| 26.6 | 30.1 | | 26.9 | 28 | | 40.5 | 37.6 | 36.4 | 34.8 | 35.9 | | 36.2 | 34.9 | |
| 26.9 | 28.0 | | 31.3 | 26.9 | | 40.6 | 38.3 | 40.7 | 35.4 | 36.2 | | 35.0 | 36.6 | |
| 28.5 | | | 28.4 | 25.7 | | | | 38.0 | | | | 43.2 | | |
| 29.3 | | | 28.8 | | | | | | | | | 34.7 | | |
| 30.3 | | | 27.8 | | | | | | | | | 37.1 | | |
| 27.5 | | | 32.1 | | | | | | | | | 42.1 | | |
| 28.2 | | | 29.2 | | | | | | | | | 32.6 | | |
| 11 | | | 12 | | | 13 | | | 14 | | | 15 | | |
| S1 | S2 | S3 | S1 | S2 | S3 | S1 | S2 | S3 | S1 | S2 | S3 | S1 | S2 | S3 |
| 27.2 | 31.8 | | 28.2 | 26.2 | | 32.2 | 30.8 | | 41.2 | 33.3 | | 27.0 | 23.0 | |
| 26.7 | 27.7 | | 28.7 | 31.4 | | 32.1 | 28.1 | | 38.8 | 32.3 | | 28.3 | 23.9 | |
| 28.2 | 27.1 | | 28.8 | 28.0 | | 29.4 | 27.9 | | 41.3 | 33.3 | | 22.0 | 26.9 | |
| 28.9 | 26.5 | | 31.0 | 26.3 | | 27.7 | 28.4 | | 43.2 | 33.9 | | 23.4 | 26.3 | |
| 27.6 | 31.0 | | 29.9 | 27.9 | | 30.1 | 29.1 | | 38.3 | 33.9 | | 25.6 | 25.4 | |
| | 29.0 | | | 26.1 | | 28.1 | | | | | | 24.5 | 24.1 | |
| | 27.7 | | | 32.7 | | 28.9 | | | | | | 26.6 | 23.0 | |
| | 27.5 | | | 25.8 | | 30.1 | | | | | | 27.9 | 24.4 | |
| | 27.3 | | | 29.0 | | 31.9 | | | | | | 25.1 | 24.4 | |
| | 29.8 | | | 29.4 | | 26.6 | | | | | | 24.2 | 25.3 | |
| 16 | | | 17 | | | 18 | | | 19 | | | 20 | | |
| S1 | S2 | S3 | S1 | S2 | S3 | S1 | S2 | S3 | S1 | S2 | S3 | S1 | S2 | S3 |
| 30.8 | 31.4 | | 27.6 | 25.3 | | 31.8 | 24.9 | | 28.1 | 26.2 | | 30.8 | 26.8 | |
| 28.1 | 30.7 | | 25.1 | 26.3 | | 27.7 | 27.0 | | 29.3 | 31.4 | | 28.1 | 33.2 | |
| 28.4 | 28.8 | | 24.6 | 28.1 | | 27.1 | 28.3 | | 30.3 | 28.0 | | 27.9 | 27.8 | |
| 27.3 | 32.8 | | 25.7 | 24.6 | | 26.5 | 28.8 | | 30.7 | 26.3 | | 28.4 | 31.6 | |
| 27.3 | 29.7 | | 25.8 | 27.4 | | 31.0 | 23.6 | | 30.6 | 27.9 | | 29.1 | 33.4 | |
| | | | | | | 29.0 | 22.4 | | | 26.1 | | | 34.5 | |
| | | | | | | 27.7 | 22.4 | | | 32.7 | | | 29.4 | |
| | | | | | | 27.5 | 28.4 | | | 25.8 | | | 29.8 | |
| | | | | | | 27.3 | 29.0 | | | 29.0 | | | 31.4 | |
| | | | | | | 29.8 | 26.1 | | | 29.4 | | | 32.6 | |

Measurements after 2 h

| 01 | | | 02 | | | 03 | | | 04 | | | 05 | | |
|---|---|---|---|---|---|---|---|---|---|---|---|---|---|---|
| S1 | S2 | S3 | S1 | S2 | S3 | S1 | S2 | S3 | S1 | S2 | S3 | S1 | S2 | S3 |
| 33.3 | 42.4 | | 43.4 | 54.6 | 65.5 | 54.3 | 38.2 | | 32.0 | 47.9 | | 50.8 | 36.4 | |
| 34.4 | 45.5 | | 42.1 | 58.2 | 56.5 | 53.5 | 42.0 | | 36.2 | 50.2 | | 50.8 | 32.9 | |
| 37.3 | 46.6 | | 41.1 | 61.4 | 63.0 | 54.1 | 43.4 | | 30.9 | 52.6 | | 51.4 | 35.9 | |
| 34.7 | 48.7 | | 41.0 | 62.0 | 60.1 | 53.4 | 42.3 | | 35.4 | 50.0 | | 53.7 | 31.8 | |
| 34.7 | 52.3 | | 41.8 | 61.5 | 61.5 | 52.4 | 37.6 | | 32.0 | 55.0 | | 57.4 | 33.3 | |
| | 47.8 | | | | | | 36.9 | | 31.2 | | | | | |
| | 49.4 | | | | | | 39.1 | | 30.9 | | | | | |
| | 44.1 | | | | | | 36.9 | | 34.0 | | | | | |
| | 50.2 | | | | | | 36.5 | | 31.3 | | | | | |
| | 50.6 | | | | | | 37.6 | | 29.7 | | | | | |
| 06 | | | 07 | | | 08 | | | 09 | | | 10 | | |
| S1 | S2 | S3 | S1 | S2 | S1 | S2 | S3 | S1 | S2 | S1 | S2 | S3 | S1 | S2 |
| 33.0 | 40.5 | | 44.3 | 28.4 | | 41.7 | 49.5 | 60.8 | 49.8 | 36.7 | | 37.3 | 42.9 | |
| 32.1 | 41.3 | | 48.4 | 28.8 | | 39.0 | 51.3 | 52.3 | 46.5 | 34.0 | | 38.0 | 45.9 | |
| 31.9 | 46.1 | | 51.8 | 27.5 | | 38.0 | 46.5 | 49.6 | 47.7 | 34.1 | | 36.2 | 48.9 | |
| 31.4 | 40.6 | | 53 | 26.2 | | 43.1 | 49.9 | 49.3 | 50.4 | 33.9 | | 34.7 | 55.2 | |
| 32.3 | 50.3 | | 53.9 | 29.6 | | 41.0 | 51.5 | 57.2 | 46.3 | 37.2 | | 35.6 | 52.0 | |
| | 49.7 | | 54.4 | | | | | 60.2 | | | | | 45.9 | |
| | 41.6 | | 52.6 | | | | | 61.1 | | | | | 49.4 | |
| | 47.8 | | 52.8 | | | | | 54.0 | | | | | 57.8 | |
| | 49.3 | | 53.4 | | | | | 51.2 | | | | | 51.2 | |
| | 50.0 | | 46.4 | | | | | 58.3 | | | | | 54.9 | |
| 11 | | | 12 | | | 13 | | | 14 | | | 15 | | |
| S1 | S2 | S3 | S1 | S2 | S1 | S2 | S3 | S1 | S2 | S1 | S2 | S3 | S1 | S2 |
| 39.5 | 27.5 | | 45.5 | 28.9 | | 40.1 | 27.9 | | 36.7 | 48.4 | | 33.1 | 23.9 | |
| 42.8 | 26.4 | | 46.1 | 34.6 | | 40.5 | 28.4 | | 34.0 | 51.9 | | 34.9 | 22.5 | |
| 43.4 | 28.3 | | 45.9 | 32.6 | | 38.7 | 31.5 | | 32.9 | 53.7 | | 31.9 | 25.1 | |
| 43.2 | 30.6 | | 46.3 | 28.7 | | 43.5 | 31.9 | | 36.5 | 55.9 | | 34.1 | 23.6 | |
| 41.8 | 27.2 | | 43.3 | 34.9 | | 38.9 | 26.5 | | 36.3 | 59.1 | | 36.9 | 25.6 | |
| | | | | 30.5 | | | 28.6 | | | 56.8 | | | | |
| | | | | 30.7 | | | 29.6 | | | 52.5 | | | | |
| | | | | 33.8 | | | 30.0 | | | 52.7 | | | | |
| | | | | 32.1 | | | 29.3 | | | 49.2 | | | | |
| | | | | 31.8 | | | 30.5 | | | 53.7 | | | | |
| 16 | | | 17 | | | 18 | | | 19 | | | 20 | | |
| S1 | S2 | S3 | S1 | S2 | S1 | S2 | S3 | S1 | S2 | S1 | S2 | S3 | S1 | S2 |
| 30.5 | 46.6 | | 46.5 | 32.5 | | 27.5 | 35.8 | | 43.8 | 28.9 | | 27.9 | 40.2 | |
| 29.6 | 45.4 | | 40.7 | 30.9 | | 26.4 | 39.8 | | 42.2 | 34.6 | | 28.4 | 41.8 | |
| 28.4 | 46.1 | | 48.4 | 29.8 | | 28.3 | 38.0 | | 45.5 | 32.6 | | 31.5 | 45.8 | |
| 31.4 | 51.2 | | 47.7 | 32.6 | | 30.6 | 32.7 | | 48.2 | 28.7 | | 31.9 | 42.0 | |
| 29.5 | 49.4 | | 46.8 | 29.4 | | 27.2 | 33.8 | | 50.6 | 34.9 | | 26.5 | 43.1 | |
| | | | 41.1 | | | | 38.2 | | 50.7 | 30.5 | | 28.6 | | |
| | | | 39.3 | | | | 32.5 | | 47.3 | 30.7 | | 29.6 | | |
| | | | 44.8 | | | | 32.5 | | 51.1 | 33.8 | | 30.0 | | |
| | | | 47.6 | | | | 34.4 | | 52.0 | 32.1 | | 29.3 | | |
| | | | 49.1 | | | | 36.9 | | 52.7 | 31.8 | | 30.5 | | |

Measurements after 5 h

| 01 | | | 02 | | | 03 | | | 04 | | | 05 | | |
|---|---|---|---|---|---|---|---|---|---|---|---|---|---|---|
| S1 | S2 | S3 | S1 | S2 | S3 | S1 | S2 | S3 | S1 | S2 | S3 | S1 | S2 | S3 |
| 34.0 | 39.1 | | 37.0 | 48.8 | 52.3 | 56.4 | 35.4 | | 31.7 | 52.2 | | 48.8 | 34.4 | |
| 34.8 | 42.5 | | 39.9 | 51.9 | 48.3 | 53.5 | 39.1 | | 33.8 | 53.2 | | 51.9 | 38.6 | |
| 37.7 | 42.1 | | 38.5 | 51.2 | 51.8 | 49.8 | 34.9 | | 31.9 | 50.2 | | 51.1 | 40.0 | |
| 37.2 | 43.4 | | 42.0 | 53.5 | 50.3 | 52.3 | 37.4 | | 35.6 | 54.3 | | 51.0 | 37.8 | |
| 34.3 | 44.6 | | 40.9 | 55.2 | 56.0 | 55.0 | 36.5 | | 32.4 | 54.4 | | 53.6 | 34.0 | |
| | | | | | | | | | | | | | 34.7 | |
| | | | | | | | | | | | | | 37.8 | |
| | | | | | | | | | | | | | 34.4 | |
| | | | | | | | | | | | | | 39.1 | |
| | | | | | | | | | | | | | 34.9 | |
| 06 | | | 07 | | | 08 | | | 09 | | | 10 | | |
| S1 | S2 | S3 | S1 | S2 | S1 | S2 | S3 | S1 | S2 | S1 | S2 | S3 | S1 | S2 |
| 34.2 | 47.2 | | 41.6 | 26.7 | | 37.3 | 43.1 | 52.6 | 49.5 | 36.4 | | 33.3 | 43.0 | |
| 30.0 | 46.9 | | 44.6 | 27.5 | | 37.0 | 43.0 | 47.4 | 48.6 | 32.1 | | 32.8 | 46.8 | |
| 29.1 | 47.6 | | 46.5 | 27.6 | | 35.7 | 48.5 | 46.7 | 48.1 | 32.9 | | 33.0 | 51.3 | |
| 34.1 | 51.4 | | 46.1 | 28.5 | | 37.9 | 46.9 | 55.0 | 47.1 | 35.8 | | 38.0 | 49.7 | |
| 29.3 | 52.0 | | 48.4 | 28.1 | | 38.4 | 47.0 | 52.9 | 45.1 | 33.9 | | 34.6 | 52.5 | |
| 30.6 | | | | | | | | 54.5 | | | | 30.0 | 50.4 | |
| 29.9 | | | | | | | | 48.2 | | | | 32.9 | 47.6 | |
| 32.8 | | | | | | | | 48.5 | | | | 33.9 | 47.2 | |
| 29.6 | | | | | | | | 51.3 | | | | 34.9 | 54.2 | |
| 32.3 | | | | | | | | 53.8 | | | | 35.1 | 49.9 | |
| 11 | | | 12 | | | 13 | | | 14 | | | 15 | | |
| S1 | S2 | S3 | S1 | S2 | S1 | S2 | S3 | S1 | S2 | S1 | S2 | S3 | S1 | S2 |
| 40.4 | 28.2 | | 40.5 | 28.5 | | 37.8 | 27.0 | | 37.0 | 46.7 | | 36.4 | 25.2 | |
| 38.8 | 25.0 | | 40.2 | 33.9 | | 37.4 | 29.2 | | 37.4 | 44.9 | | 34.6 | 22.3 | |
| 40.1 | 31.7 | | 42.0 | 28.9 | | 35.6 | 30.7 | | 34.4 | 47.8 | | 38.3 | 26.1 | |
| 39.8 | 33.1 | | 44.1 | 30.6 | | 39.3 | 29.3 | | 34.4 | 48.7 | | 35.4 | 26.5 | |
| 43.1 | 27.0 | | 41.2 | 35.4 | | 43.3 | 28.8 | | 37.5 | 49.1 | | 38.8 | 24.4 | |
| | 27.0 | | | 30.8 | | 41.0 | | | | | | | 28.6 | |
| | 28.2 | | | 29.2 | | 38.3 | | | | | | | 24.0 | |
| | 27.3 | | | 30.7 | | 39.7 | | | | | | | 26.1 | |
| | 28.4 | | | 31.2 | | 34.0 | | | | | | | 27.0 | |
| | 28.1 | | | 33.8 | | 40.3 | | | | | | | 25.8 | |
| 16 | | | 17 | | | 18 | | | 19 | | | 20 | | |
| S1 | S2 | S3 | S1 | S2 | S1 | S2 | S3 | S1 | S2 | S1 | S2 | S3 | S1 | S2 |
| 26.7 | 47.1 | | 35.9 | 31 | | 28.2 | 42.6 | | 42.7 | 28.5 | | 27.0 | 33.5 | |
| 27.1 | 45.7 | | 32.8 | 28.6 | | 25.0 | 43.7 | | 44.5 | 33.9 | | 29.2 | 37.9 | |
| 29.7 | 48.4 | | 30.6 | 24.9 | | 31.7 | 41.3 | | 45.1 | 28.9 | | 30.7 | 40.1 | |
| 27.8 | 51.2 | | 38.3 | 26.7 | | 33.1 | 44.0 | | 47.2 | 30.6 | | 29.3 | 40.6 | |
| 30.7 | 49.0 | | 37.8 | 30.7 | | 27.0 | 40.8 | | 48.8 | 35.4 | | 28.8 | 39.0 | |
| 24.4 | | | 36.6 | 26.9 | | 27.0 | | | | 30.8 | | | 33.6 | |
| 30.7 | | | 36.1 | 28.4 | | 28.2 | | | | 29.2 | | | 42.2 | |
| 27.9 | | | 33.9 | 27.3 | | 27.3 | | | | 30.7 | | | 39.4 | |
| 27.7 | | | 39.3 | 30.4 | | 28.4 | | | | 31.2 | | | 40.5 | |
| 29.2 | | | 39.5 | 27.8 | | 28.1 | | | | 33.8 | | | 40.8 | |

Measurements after 8 h

| 01 | | | 02 | | | 03 | | | 04 | | | 05 | | |
|---|---|---|---|---|---|---|---|---|---|---|---|---|---|---|
| S1 | S2 | S3 | S1 | S2 | S3 | S1 | S2 | S3 | S1 | S2 | S3 | S1 | S2 | S3 |
| 33.0 | 38.7 | | 38.7 | 46.4 | 48.3 | 52.2 | 34.2 | | 31.0 | 46.3 | | 43.2 | 35.7 | |
| 35.0 | 39.0 | | 37.9 | 48.2 | 48.5 | 51.2 | 34.4 | | 30.2 | 49.6 | | 38.5 | 34.3 | |
| 38.3 | 38.3 | | 40.0 | 47.8 | 55.2 | 51.3 | 35.6 | | 30.7 | 49.1 | | 39.9 | 34.7 | |
| 32.9 | 39.3 | | 41.6 | 49.5 | 50.2 | 54.9 | 33.4 | | 35.0 | 51.4 | | 43.9 | 32.9 | |
| 36.5 | 41.4 | | 41.5 | 50.5 | 50.4 | 55.5 | 34.7 | | 33.0 | 54.6 | | 45.4 | 37.1 | |
| 32.0 | | | | | | | | | 34.3 | 48.8 | | 47.0 | | |
| 34.1 | | | | | | | | | 32.7 | | | 47.5 | | |
| 33.4 | | | | | | | | | 34.0 | | | 49.9 | | |
| 34.4 | | | | | | | | | 30.8 | | | 41.0 | | |
| 35.0 | | | | | | | | | 31.7 | | | 46.7 | | |
| 06 | | | 07 | | | 08 | | | 09 | | | 10 | | |
| S1 | S2 | S3 | S1 | S2 | S1 | S2 | S3 | S1 | S2 | S1 | S2 | S3 | S1 | S2 |
| 32.7 | 41.3 | | 41.6 | 29.3 | | 37.9 | 44.2 | 52.7 | 46.3 | 34.6 | | 37.3 | 44.6 | |
| 29.1 | 41.9 | | 44.6 | 30.1 | | 37.5 | 43.8 | 53.8 | 44.0 | 34.3 | | 38.5 | 48.5 | |
| 29.9 | 40.9 | | 44.2 | 28.4 | | 39.6 | 42.5 | 44.5 | 43.9 | 33.5 | | 36.1 | 51.1 | |
| 29.5 | 40.3 | | 44.4 | 30.1 | | 37.5 | 46.9 | 46.2 | 42.3 | 35.4 | | 34.2 | 46.1 | |
| 31.0 | 43.5 | | 45.1 | 33 | | 39.9 | 44.5 | 43.8 | 41.8 | 33.9 | | 33.5 | 54.4 | |
| | | | | | | | | 49.3 | | | | | 51.0 | |
| | | | | | | | | 49.3 | | | | | 50.1 | |
| | | | | | | | | 52.2 | | | | | 49.7 | |
| | | | | | | | | 51.9 | | | | | 52.9 | |
| | | | | | | | | 50.5 | | | | | 55.6 | |
| 11 | | | 12 | | | 13 | | | 14 | | | 15 | | |
| S1 | S2 | S3 | S1 | S2 | S1 | S2 | S3 | S1 | S2 | S1 | S2 | S3 | S1 | S2 |
| 41.0 | 26.5 | | 46.7 | 28.5 | | 37.4 | 28.6 | | 39.2 | 45.4 | | 40.2 | 27.0 | |
| 39.6 | 26.8 | | 44.2 | 32.3 | | 39.0 | 29.7 | | 37.2 | 45.8 | | 32.7 | 26.3 | |
| 40.0 | 30.8 | | 46.0 | 33.8 | | 39.6 | 29.6 | | 36.3 | 45.3 | | 38.9 | 25.3 | |
| 40.7 | 28.3 | | 41.5 | 33.8 | | 40.9 | 32.3 | | 37.7 | 45.7 | | 35.7 | 27.5 | |
| 38.6 | 30.9 | | 50.8 | 29.2 | | 41.2 | 28.4 | | 38.0 | 46.2 | | 32.5 | 29.2 | |
| | 27.8 | | 48.0 | 28.9 | | | | | | | | 38.2 | | |
| | 28.6 | | 47.8 | 30.7 | | | | | | | | 32.6 | | |
| | 27.9 | | 52.7 | 33.2 | | | | | | | | 36.0 | | |
| | 28.4 | | 46.5 | 30.5 | | | | | | | | 36.1 | | |
| | 30.2 | | 53.1 | 35.3 | | | | | | | | 32.8 | | |
| 16 | | | 17 | | | 18 | | | 19 | | | 20 | | |
| S1 | S2 | S3 | S1 | S2 | S1 | S2 | S3 | S1 | S2 | S1 | S2 | S3 | S1 | S2 |
| 31.0 | 43.8 | | 33.9 | 34.3 | | 26.5 | 36.6 | | 45.9 | 28.5 | | 28.6 | 42.1 | |
| 28.7 | 40.0 | | 32.8 | 31.6 | | 26.8 | 34.3 | | 42.1 | 32.3 | | 29.7 | 41.5 | |
| 30.9 | 43.8 | | 36.4 | 28.2 | | 30.8 | 30.6 | | 46.8 | 33.8 | | 29.6 | 40.8 | |
| 29.6 | 45.5 | | 33.1 | 27.1 | | 28.3 | 36.3 | | 44.4 | 33.8 | | 32.3 | 40.8 | |
| 29.8 | 42.9 | | 37.3 | 27.5 | | 30.9 | 33.1 | | 50.5 | 29.2 | | 28.4 | 44.2 | |
| | | | | 31.9 | | 27.8 | 36.1 | | 47.6 | 28.9 | | | | |
| | | | | 26.9 | | 28.6 | 33.5 | | 46.9 | 30.7 | | | | |
| | | | | 28.7 | | 27.9 | 32.6 | | 44.9 | 33.2 | | | | |
| | | | | 29.6 | | 28.4 | 36.4 | | 47.8 | 30.5 | | | | |
| | | | | 31.2 | | 30.2 | 33.5 | | 48.6 | 35.3 | | | | |

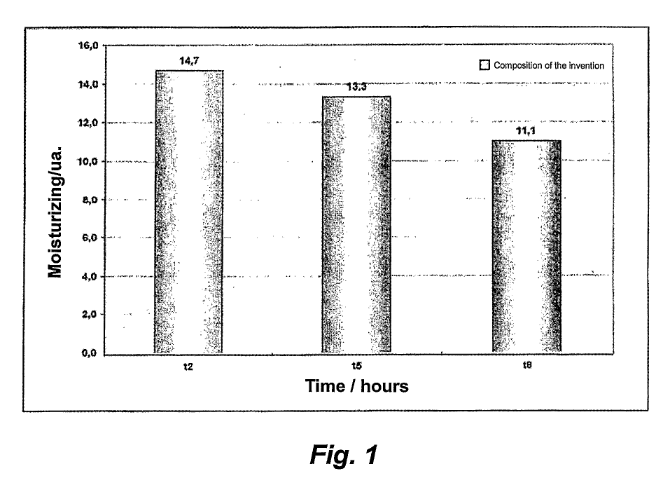

The data measured on the Corneometer® were analyzed by using the software Microsoft® Office Excel 2003. From the average values measured on the Corneometer®, the skin moisturizing imparted by the application of the composition was evaluated by using the equations 1 and 2, the results of which are presented in FIG. 1, which refers to a graph demonstrating the moisturizing imparted to the skin in the forearm region throughout the time by the composition of the invention with respect to the control (without product).

Equation 1: Moisturizing difference between the reading for the Control and the Composition of the invention or Standard Product in each time

Δh=hₜᵢ−hₜ₀

wherein:

Δh=moisturizing difference

hₜᵢ=average of the readings in the area X in time ti

hₜ₀=average of the readings in the area X in t0

Equation 2: Calculation of the moisturizing

H=Δh_{ti(product)}−Δh_{ti(control)}

wherein:

H=skin moisturizing in the area X after the times ti of application of the product:

Δh_{ti(product)} e Δh_{ti(control)}=difference in moisturizing intensity for the control and the product with respect to the basal reading in time ti, respectively.

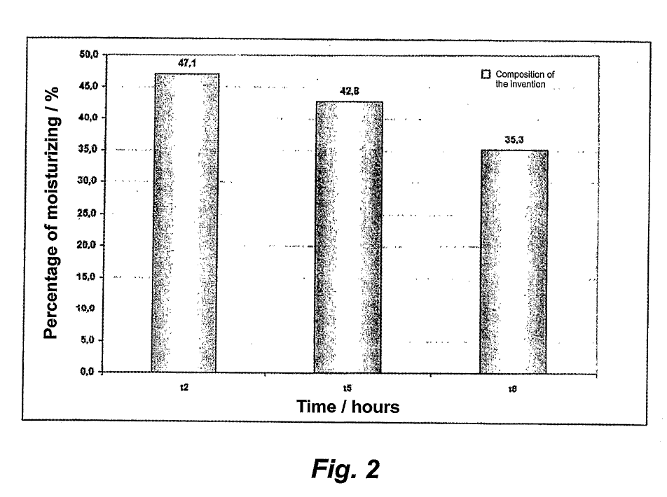

FIG. 2 presents the kinetics of the moisturizing percentage of the composition of the invention calculated by using the Equation 3, that is to say, the moisturizing percentage imparted to the skin in the forearm region throughout the time by the composition of the invention with respect to the control (skin without product).

Equation 3: Calculation of the moisturizing percentage in the time ti

% moisturizingₜᵢ=(Hₜᵢ×100)/hₜ₀

where:

Hₜᵢ=skin moisturizing in the area X after the times ti of application of the product;

hₜₒ=average of the basal readings in the area X in t0;

Concomitant with the product evaluated for the invention, tests with the Standard Product were carried out, obtaining the following average moisturizing values (n=2), with reliability interval 95%: moisturizing 2 h: 19.3 ua; moisturizing 5 h: 15.1 ua and moisturizing 8 h: 12.9 ua. These results are in accordance with the reliability intervals 95% foreseen in the validation: moisturizing 2 h: reliability interval 95%: 16.5 ua to 19.7 ua; moisturizing 5 h: reliability interval 95%: 10.7 ua to 15.5 ua and moisturizing: reliability interval 95%: 9.0 ua to 13.0 ua.

Conclusion: The application of the composition of the invention onto the skin in the forearm region provided statistically significant moisturizing when compared with the control (skin without application of any products) until 8 hours after application.

### Test 03—Affective Sensorial Study

This test has the objective of evaluating the acceptance of the consumers (Affective Evaluation) through the test of monadic domiciliary use.

It were selected 86 women with ages between 18 and 50 years, who supported a line of bath products that did not leave the skin so oily, belonging to the classes A and B. The exclusion criteria comprised: history of adverse reactions to the use of a moisturizer and body oil, with sinusitis, rhinitis, allergy to perfumes, pregnant or breast-feeding women.

On the first day of test, the volunteers were informed of the conditions of the test, discriminated below:

using the cosmetic composition of the present invention during the bath, at least three times during the 10 days. Writing down the number of times that the product was used and

not using the moisturizer and the body oil of customary use, during the period of test.

### Results Achieved:

### 1. Global Evaluation

84.3% of the volunteers liked the product as a whole

### 2. Consistency

86.7% of the volunteers liked the consistency of the product.

68.7% of the volunteers said that the consistency of the product was ideal.

### 3. Ease of Application

83.1% of the volunteers liked the application of the product.

78.3% of the volunteers said that the application was easy.

### 4. Ease of Rinsing

91.6% of the volunteers liked the ease of rinsing of the product;

91.6% of the volunteers said that the rinsing was easy.

### 5. Stickiness

75.9% of the volunteers liked the stickiness of the product;

86.7% of the volunteers said that the skin did not become sticky.

### 6. Oiliness of the Skin After Using the Product

79.5% of the volunteers liked the oiliness of the skin after using the product;

77.1% of the volunteers said that the skin did not become oily.

### 7. Moisturizing/Softness Sensation

75.9% of the volunteers liked the moisturizing/softness sensation of the skin after using the product;

55.4% of the volunteers said that the skin became softer/more moisturized than usual.

### 8. Velvety Touch Sensation (Peach Skin)

60.2% of the volunteers said that the products had promoted a velvety-touch feeling.

### 9. Appearance of the Treated Skin

61.4% of the volunteers noted an appearance of treated skin after using the product.

### 10. Drying

80.7% of the volunteers said that the skin did not become dry after using the product.

1. A multifunctional cosmetic oil-in-water emulsion, characterized by comprising:
a silicone system composed by cyclomethicone as a light silicone present in an amount of 4.00% by weight, and a mixture of cyclomethicone and dimethiconol as a heavy silicone present in an amount of 2.00% by weight, and
an emollient system composed by at least one component selected from cupuaçu butter, murumuru butter and Shea butter in an amount ranging from 1.0% to 5.0% by weight,
a cosmetically acceptable carrier,
wherein all the amounts based on the total weight of the cosmetic composition.

2. A cosmetic composition according to claim 1, characterized in that the emollient system comprises cupuaçu butter in an amount of 2.0% by weight, and murumuru butter in an amount of 2.0 by weight, based on the total weight of the composition.

3. A cosmetic composition according to claim 2, characterized in that the emollient system comprises decyl oleate, dicaprylic ether and cetyl lactate.

4. A cosmetic composition according to claim 3, characterized in that the emollient system comprises cupuaçu butter in an amount of 1.0% by weight, and murmmuru butter in an amount of 1.0% by weight, based on the total weight of the composition.

5. A cosmetic composition according to claim 1, characterized by comprising a refreshing agent.

6. A cosmetic composition according to claim 5, characterized in that the refreshing agent is mentol present in an amount of 0.10% by weight, based on the total weight of the composition.

7. A cosmetic composition according to claim 1, characterized by comprising an emulsifying system composed by stearet-2, stearet-21 and glyceryl stearate and in an amount ranging from 3.0% to 5.0% by weight, based on the total weight of the composition.

8. A process for preparing the cosmetic composition as defined in claim 1, characterized by comprising the following steps:
Preparation of Phase A
adding to a main recipient a cosmetically acceptable carrier;
Preparation of Phase D
initiating heating until a temperature ranging from 75° C. to 80° C. is reached;
Preparation of Phase E
with the temperature between 75° C. and 80° C., solubilizing at least one butter selected from cupuaçu butter, murumuru butter and Shea butter, maintaining the temperature between 75° and 80° C., under high stirring ranging from 30 rpm to 3000 rpm until total homogenization is achieved;
Preparation of Phase F
initiating cooling until a temperature between 55° C. and 60° C. is reached, and adding the cyclomethicone and the mixture of cyclomethicone and dimethiconol.

9. A process according to claim 8, characterized by comprising the following additional step:
Preparation of Phase B
adding a suspending thickening agent to the phase A at a temperature of about 25° C., mixing the composition at a frequency ranging from 2500 to 3500 rpm until total solubilization of the polymer thickening agent is achieved.

10. A process according to claim 8, characterized by comprising the following additional step:
Preparation of Phase C:
adding a moistening agent to the phase A or phase C at a temperature of 25° C.

11. A process according to claim 8, characterized by comprising the following additional step:
Preparation of Phase G:
in a separate recipient, solubilizing a neutralizing agent in the carrier and pouring onto the main recipient, after carrying out Phase F, thus neutralizing the emulsion, at a temperature between 50° C. and 55° C., reaching total homogenization and neutralization.

12. A process according to claim 11, characterized by comprising the following additional step:
Preparation of Phase H:
after carrying out Phase G, when the temperature of 45° C. is reached, adding a polymeric consistency agent to the main recipient to form a cream gel.

13. A process according to claim 12, characterized by comprising the following additional step:
Preparation of Phase I
after carrying out Phase H, when a temperature between 30° C. and 35° C. is reached, adding at least one preservative agent, a fragrance and at least one dye to the main recipient and keep stirring until a temperature of 25° C. is reached.

14. A multifunctional cosmetic composition characterized by being obtainable by the preparation process as defined in claim 8.

15. A cosmetic product characterized by comprising the cosmetic composition as defined in claim 1.

16. A cosmetic product characterized by comprising the cosmetic composition as defined in claim 15.
